(19) United States
(12) Patent Application Publication
(10) Pub. No.: US 2020/0101016 A1
(43) Pub. Date: Apr. 2, 2020

(54) COMPOSITIONS AND METHODS FOR PRODUCING EXOSOME LOADED THERAPEUTICS FOR TREATING CARDIOVASCULAR DISEASE

(71) Applicant: Exosome Therapeutics, Inc., San Diego, CA (US)

(72) Inventors: Gerardo Rodriguez-Araujo, San Diego, CA (US); Stephen R. Puckett, SR., Charlotte, NC (US); Stephen R. Puckett, JR., Charolotte, NC (US); Mitchell W. Puckett, Charolotte, NC (US)

(73) Assignee: Exosome Therapeutics, Inc., San Diego, CA (US)

(21) Appl. No.: 16/591,483

(22) Filed: Oct. 2, 2019

Related U.S. Application Data
(60) Provisional application No. 62/740,396, filed on Oct. 2, 2018.
(60) Provisional application No. 62/740,382, filed on Oct. 2, 2018.
(60) Provisional application No. 62/740,391, filed on Oct. 2, 2018.

(51) Int. Cl. A61K 9/127; C12N 15/85; A61K 48/00; C07K 16/18; A61K 35/28; C12N 15/113

(52) U.S. Cl. CPC A61K 9/1271; C12N 15/85; A61K 48/0066; C07K 16/18; C12N 2310/14; C12N 15/113; C12N 2015/8518; C07K 2317/76; A61K 35/28

(57) ABSTRACT

A composition for delivering cargo to cytoplasm of a cell, wherein the cargo manipulates angiogenesis. In one embodiment the composition comprises: an exosome; and cargo, located within the exosome, comprising at least one plasmid. In another embodiment the composition comprises: an exosome; and cargo, located within the exosome, comprising a DNA plasmid bioengineered specifically to self-produce monoclonal neutralizing antibodies.

### CROSS-REFERENCE TO RELATED APPLICATIONS

The present application claims priority to U.S. Provisional Patent Application Nos. 62/740,396 filed Oct. 2, 2018, entitled “METHODS OF PRODUCING cGMP GRADE AND RESEARCH ONLY GRADE AUTOLOGOUS AND ALLOGENIC EXOSOMES AS CARRIERS FOR THERAPEUTIC COMPOUNDS FOR USE IN HUMANS AND IN PRECLINICAL STUDIES IN ANIMALS;” 62/740,382 filed Oct. 2, 2018, entitled “cGMP GRADE AND RESEARCH ONLY GRADE AUTOLOGOUS EXOSOMES FOR THE PROMOTION (USING VASCULAR ENDOTHELIAL GROWN FACTOR 1, 2 AND 3) OR REPRESSION OF ANGIOGENESIS (ANTI-MIR-214) FOR USE IN HUMANS AND IN PRECLINICAL STUDIES IN ANIMALS;” and 62/740,391 filed Oct. 2, 2018, “cGMP GRADE AND RESEARCH ONLY GRADE AUTOLOGOUS EXOSOMES FOR THE PRIMARY AND/OR SECONDARY PREVENTION OF CARDIOVASCULAR DISEASE (CVD), INFLAMMATION AND THEIR ASSOCIATED COMPLICATIONS USING PCSK-9, IL-1B, IL4 AND 13 AS THERAPEUTIC TARGETS FOR USE IN HUMANS AND IN PRECLINICAL STUDIES IN ANIMALS,” the entire disclosures of which are incorporated by reference herein.

### BACKGROUND OF THE INVENTION

### 1. Field of Invention

This invention relates to extracellular vesicles for therapeutic delivery, and more specifically, to compositions and methods of producing exosomes comprising therapeutics to promote or repress angiogenesis that meet good manufacturing practices standards for use in treating cardiovascular diseases and cancer in humans and in animals.

### 2. Description of Related Art

Cardiovascular Disease (CVD) is a leading cause of human deaths worldwide. It consists of a series of inflammatory and atherogenic-lipid mechanisms (forming fatty plaques) that reduce blood vessel diameter and, subsequently, reduce the amount of blood oxygen and nutrients accessible to tissues. Additionally, in areas of lipid accumulation (atherosclerotic plaques), it has been postulated that the oxidation of low-density lipoprotein (LDL) triggers inflammatory responses of interleukins, (e.g., IL 1, 6, 4, 13, tumor necrosis factor alpha (TNFα), etc.) which leads to deleterious wound healing processes in the atherosclerotic plaque. Further, rupture or instability of the plaque can trigger acute occlusive and thrombotic events that lead to distal ischemia or infarction.

Current pharmacotherapy methods, for treating CVD or related complications, are limited in efficacy in patients and further by drug costs. Lipid or cholesterol lowering medications such as statins and fibrates are only partially efficient in a reduced number of patients, having neutral or null effects on many cases. Additionally, statins commonly precipitate myalgias and body aches as the mechanism of action negatively affects myocytes biology. Proprotein convertase subtilisin/kexin type 9 (PCSK9) inhibitors represent a new generation of lipid-lowering, anti-atherogenic, drugs. The high drug costs, however, preclude patients from using PCSK9 inhibitors. Preparation and delivery methods using exosomes containing RNA or DNA plasmids which secret human anti-PC SK9 antibodies has never been taught by the prior art. By using this new method of exosome-mediated delivery, anti-PCSK9 antibodies are distributed much more efficiently and effectively to the target location. This novel therapeutic combination represents a low cost and highly efficient agent to inhibit PCSK9 and atherosclerosis as well as its inflammatory component by inhibiting inflammatory responses from interleukins.

The PCSK9 gene on chromosome 1 in humans provides instructions for making a PCSK9 protein that helps regulate the amount of cholesterol in the bloodstream. The PCSK9 protein appears to control the number of low-density lipoprotein receptors, which are proteins on the surface of cells that regulate blood cholesterol levels through binding LDLs carrying cholesterol in the blood. Hence, an approach to lowering blood cholesterol in CVD is to efficiently and affordably deliver PCSK9 inhibitors to patients.

Ray et al. teaches that in patients with a high risk of CVD who had elevated LDL cholesterol levels were given inclisiran, a chemically synthesized small interfering RNA (siRNA) designed to target PCSK9 messenger RNA (mRNA), at varying dosage levels (Ray et al. Inclisiran in Patients at High Cardiovascular Risk with Elevated LDL Cholesterol. N Engl J Med 2017; 376(15): 1430-1440). Patients who received a subcutaneous injection of inclisiran had dose-dependent reductions in the PCSK9 protein and LDL cholesterol levels. However, adverse events were reported in 76% of patients and serious adverse events occurred in 11% of patients. This prior art does not use exosome-mediated delivery of therapeutic molecules to deliver inclisiran or cGMP exosomes loaded with a PCSK9 interference RNA (iRNA).

Another approach to preventing and treating CVD has been to promote new vascular vessel growth (angiogenesis). In particular, facilitating the differentiation or trans-differentiation of cells into vascular cells and vascular tissues has been shown to be useful in treating CVD. The promotion or repression of angiogenesis has been targeted approaches for treating certain diseases and conditions. Promoting angiogenesis is particularly useful in ischemic diseases in which native vessels are occluded or sub-occluded in areas where new vessels serve bypassing native vessels to irrigate distal tissues. In particular, promoting angiogenesis is a helpful approach in treating the following diseases: CVD, coronary arterial disease, atherosclerotic CVD, peripheral arterial disease, and peripheral vascular disease. Novel vessels or new coronary collaterals can help alleviate symptoms and improve or reestablish the function of distal tissues and organs. Vascular endothelial growth factor (VEGF) is a protein that facilitates differentiation or trans-differentiation of cells into vascular cells and tissues. Current VEGF therapeutics are limited due to the lack of integration of the VEGF protein or VEGF gene into a target cell using conventional drug delivery systems.

Mathiyalagan et al. teaches that injecting exosomes from human CD34+ cells (CD34Exo), which were found to be enriched with proangiogenic microRNAs (miRNAs) including miR-126-3p. (Mathiyalagan et al. Angiogenic Mechanisms of Human CD34+ Stem Cell Exosomes in the Repair of Ischemic Hindlimb. Circ Res 2017; 120(9): 1466-1476). When CD34Exo was injected into mouse ischemic hindlimb tissue, this mimicked the beneficial activity of their parent cells by improving ischemic limb perfusion, capillary density, motor function, and their amputation. Injection with a conditioned media that depleted the exosomes (CD34Exo-depleted conditioned media) did not improve ischemic limb perfusion, capillary density, motor function, and their amputation. CD34Exo-mediated enhanced angiogenic activity resulted in a significant reduction in SPRED1 mRNA expression, a direct target of miR-126-3p, and increased expression of proangiogenic mRNAs including VEGF as wells as ANG1 (angiopoietin 1), and MMP9 (matrix metallopeptidase 9). However, this prior art only teaches that human derived CD34 exosomes can benefit myocardial and critical limb ischemia but does not suggest that purified and isolated exosomes loaded with VEGF could have a therapeutic benefit for treating CVD. Further, this prior art does not artificially load exosomes with therapeutic molecules.

A large number of illnesses are caused by metastasis growths and tumors. Repressing angiogenesis to suppress tumor growth and metastasis growth has been an approach in treating oncological diseases. In particular, the vertebrate-specific family of miRNA precursors, miR-214, has been described as a key mediator of tumor growth in multiple cancers as well as a “sensitizer” of tumor cells to respond to cisplatin and/or other chemotherapy agents. Current pharmacotherapy aims to target mechanisms of angiogenesis because tumors are metabolically hyperactive and require enormous amounts of blood oxygen and nutrient supplies, which is facilitated by angiogenesis. Inhibiting or blocking angiogenesis in tumoral cells would prevent tumor sustaining growth. Current therapies, however, are limited by deficiencies in drug delivery methods to efficiently target and deliver effective therapeutics for suppressing angiogenesis supporting tumors.

Present drug developers face challenges in using viral vectors to deliver target genetic materials to specific cells and specific tissues due to the side effects of viral transfection and eliciting undesired immune responses. Both viral and non-viral vectors have shown modest to poor results, with the caveat of inducing immune responses. Specifically, existing viral and non-viral vectors generate antibodies, anti-viruses or anti-carriers that limit the bioavailability and increase the safety profile of a potential therapeutic product (Arrighetti et al. Exosome-like nanovectors for drug delivery in cancer. Curr Med Chem (2018); Khan et al. Challenges and innovations of drug delivery in older age. Adv Drug Delivery Rev (2018)).

Extracellular vesicles called exosomes are endogenous particles found in all body compartments that are highly effective and efficient in cell communication (Arrighetti et al. Exosome-like nanovectors for drug delivery in cancer. Curr Med Chem (2018)). In particular, exosomes exist in body fluids such as blood, urine, and biological secretions. The function of exosomes is to share information between cells in a rapid and efficient manner. This cell-to-cell communication facilitates delivery and receipt of information, (e.g., genetic materials, proteins, particles, signals, etc.) which allows specific cellular microenvironments to synchronize their function and their architecture in response to any stimulus. Exosomes are relatively small and flexible particles usually between 30 to 130 nanometers in diameter and are composed of similar materials as normal endogenous cell membranes. Hence, exosomes have been used as effective drug carriers for direct delivery into the cytoplasm of cells in tissues with minimal to no adverse immune responses. Nothing in the prior art has described a preparation and delivery method of exosomes containing RNA or DNA plasmids secreting human anti-PCSK-9 antibodies. The new method of exosomes-mediated drug delivery enables anti-PCSK-9 antibodies to be distributed more efficiently and effectively to the target location compare to drug delivery without exosomes. Exosomes represent a safe, non-viral, drug delivery system in vivo to nearby or distal cells for treating disease, including CVD and oncological diseases.

In light of the described challenges, there is an unmet medical need for an improved drug delivery system using exosome-based therapeutics or diagnostics in humans. In particular, there is a need for cGMP exosome-based therapeutics to better target angiogenesis in order to treat atherosclerosis and oncogenesis in vivo.

### SUMMARY OF THE INVENTION

The present invention overcomes these and other deficiencies of the prior art by providing cGMP autologous and/or universal donor exosomes loaded with cGMP grade genetic materials in order to promote or reduce angiogenesis. The present invention describes exosome-mediated compositions that treat CVD, coronary arterial disease, atherosclerotic CVD, peripheral arterial disease, or peripheral vascular disease by lowering LDL levels or promoting angiogenesis to increase vascular vessels and to treat cancer by reducing angiogenesis to halt tumor growth.

In one embodiment, the novel therapeutic combination represents a low cost and highly efficient agent to inhibit PCSK9 and atherosclerosis. In said embodiment, therapeutic agents consisting of autologous exosomes, loaded with a secreting VEGF plasmid, are used to transduce surrounding peri-ischemic cells, which facilitates angiogenesis in the distal ischemic regions. In another embodiment, therapeutic agents consisting of autologous exosomes, loaded with anti-miR214, are used to reach and suppress angiogenesis that favors oncogenesis. Therapeutic agents, consisting of autologous exosomes loaded with anti-miR214, will have wide applications in the medical field. The present invention describes methods to produce and assemble exosomes loaded with cGMP grade genetic materials in order to promote or reduce angiogenesis. In one embodiment, the present invention provides methods, which improve upon the current drug delivery challenges by providing a method of transporting angiogenesis promoting or suppressing molecules into the cytoplasm of cells allowing for delivery directly into the cellular mechanisms. The present invention transports these molecules into the cytoplasm of cells with extremely high efficiency and with no immune reactions or antibody/auto-antibody development. In another embodiment, the present invention provides methods, which improve upon the current drug delivery challenges by providing a method of transporting atherosclerosis suppressing molecules into the cytoplasm of cell allowing for delivery directly into the cellular mechanisms.

In one embodiment, the invention comprises steps to yield cGMP grade autologous exosomes or research grade autologous exosomes from peripheral blood or any other body fluids and/or human/animal cell culture.

The advantages of the present invention include lowering LDL levels in patients with CVD to reduce the development of plaques, promoting angiogenesis in patients with CVD in order to increase vascular vessels, and reducing angiogenesis in patients with cancer in order to halt the growth of tumors. The advantages of the present invention over the prior art include reducing health care costs, increasing the efficacy of treatment through exosome delivery, and limiting immune responses from therapeutic delivery.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 illustrates a method of producing autologous exosomes from a body fluid according to an embodiment of the invention.

FIG. 2 illustrates a method of producing autologous exosomes according to another embodiment of the invention.

FIG. 3 illustrates a method of producing allogenic exosomes from a cell culture according to an embodiment of the invention.

FIG. 4 illustrates a method of producing allogenic exosomes from a body fluid according to another embodiment of the invention.

FIG. 5 illustrates a method for using exosomes comprising a therapeutic cargo for treating CVD according to one embodiment of the invention.

FIG. 6 illustrates a method for using exosomes comprising a therapeutic cargo for treating CVD according to another embodiment of the invention.

FIG. 7 illustrates a table of parameters for exosome isolation and purification according to an embodiment of the invention.

FIG. 8 illustrates an exosome loaded with cargo according to an embodiment of an invention.

FIG. 9 illustrates an exosome loaded with cargo according to an embodiment of an invention.

FIG. 10 illustrates an exosome loaded with a nuclease base editor according to an embodiment of the invention.

### DETAILED DESCRIPTION OF PREFERRED EMBODIMENTS

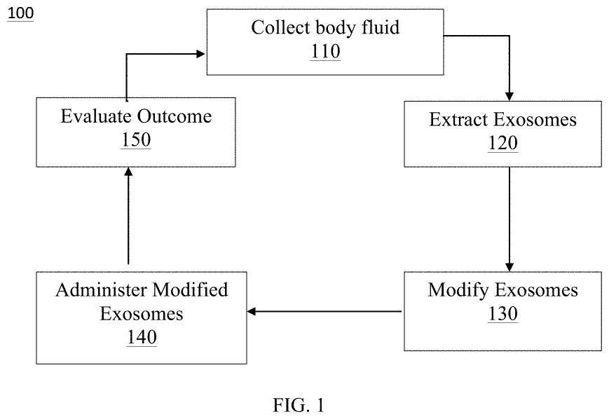

Preferred embodiments of the present invention and their advantages may be understood by referring to FIGS. 1-10. The described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents. Reference will now be made in detail to the preferred embodiments of the invention.

The term “exosome” as used herein refers to any extracellular vesicle derived from any body fluid from a human or an animal (e.g., blood), any extracellular vesicle derived from human or animal cell lines, cell cultures, and primary cultures not limited to autologous exosomes, universal donor exosomes, allogenic exosomes, and modified exosomes. In certain examples, the exosome is made to meet pharmaceutical and current good manufacturing practices (cGMP).

The term “cargo” as used herein refers to any type of molecule or any type of RNA (miRNA, mRNA, tRNA, rRNA, siRNA, iRNA, regulating RNA, gRNA, long interference RNA, non-coding and coding RNA); any type of DNA (DNA fragments, DNA plasmids, iDNA); including any type of nucleic acid including antisense oligonucleotides (ASO); any genetic material; any genetic construct; any nucleic acid construct; any plasmid or vector; any protein including recombinant endogenous protein, enzyme, antibody, wnt signaling proteins; any lipid; any therapeutic molecule or diagnostic molecule; any cellular component; chimeric antigen receptor T cell (CAR-T cell) transduced without using retroviruses; any virus including retrovirus, adenoviruses (AdV), adeno-associated viruses (AAV) of any variety and strain, and DNA viruses; any gene editing technology including clustered regularly interspaced short palindromic repeats (CRISPR), CRISPR/Cas9 system, any endonucleases for base editing, a Zinc finger, a single base editor, Transcription activator-like effector nucleases (TALENs), any meganuclease; any synthetic molecular conjugate; or combination thereof loaded into an exosome. Typically, such cargo is naturally not present in the exosome. In certain examples, the cargo is made to meet pharmaceutical and cGMP standards.

In one embodiment cargo could include a promoter. The term “promoter” as used herein refers to any DNA sequence that promotes the transcription of a gene. A plasmid comprises a tissue-specific promoter. Morever, the promoter comprises any tissue-specific promoter (e.g., lung, liver, or any other tissue type), a self-inactivating (SIN) sequence, vesicular stomatitis virus-G protein (VSV-G), or a combination thereof. The advantage of using a tissue-specific specific promoter is to better target a desired tissue in which to transcribe RNA and subsequently encode a protein.

The term “fluid” as used herein refers to any type of body fluid produced by a human or an animal including but not limited to blood, cerebral spinal fluid, urine, saliva, and any biological secretions, etc.

FIGS. 1-4 illustrate methods of producing exosomes and cargo, and methods for cargo loading into exosomes. Such improved methods and techniques would be appreciated by one of ordinary skill, especially those for increasing yield of purified exosomes and efficient loading of exosome cargo for use in preclinical and clinical studies. The methods of loading genetic material (e.g., constructs of DNA or RNA, or any type of nucleic acids) directly into exosomes are transformation, transfection and microinjection. In one embodiment, exosomes are extracted, isolated and purified from peripheral blood mononuclear cells (PBMC) circulating in peripheral blood. In such an embodiment, PBMCs are harvested from a patient or a universal donor. PBMCs are isolated and expanded in vitro using closed systems for cell culture. In another embodiment, open systems may be used depending on available resources. PBMCs produce and secrete exosomes into the media of a cell culture. The media can be filtered and exosomes can be sorted by specific parameters and purified to improve exosome quality.

The present extracellular vesicular compositions may be used to treat any of the following diseases including, but not limited to: 1. Cancer and oncological disorders including carcinogenesis, malignancies, tumors, metastasis, nodules of any variety (endodermal, mesodermal or ectodermal origin and due to spontaneous mutations or human papillomavirus or other viral infections); 2. Infectious diseases including human immunodeficiency virus and Ebola viral infections; 3. CVD including coronary arterial disease, peripheral vascular disease, peripheral arterial disease, chronic heart failure (ischemic and non-ischemic), stroke, acute kidney failure, endothelial dysfunction, mitochondrial dysfunction, oxidative stress, etc.; 4. Diabetes mellitus including Type-1 diabetes mellitus and Type-2 diabetes mellitus and any of related complications such as diabetic foot, diabetic retinopathy, peripheral diabetic neuropathy, diabetic kidney disease, insulin resistance, pre-diabetes, gestational diabetes, etc.; 5. Non-alcoholic liver disease, non-alcoholic steatohepatitis, non-alcoholic cirrhosis for primary and secondary prevention; 6. Obesity, overweightness, obesity type-1, type-2, and type 3, morbid obesity, and bariatric surgery; 7. Rare diseases; 8. Gastro-intestinal diseases including Ulcerative colitis, Crohn's disease, etc.; 9. Musculo-skeletal diseases. Further, the present extracellular vesicular compositions may be used for cell therapeutics, vector and cell engineering, pharmacology and toxicology assay development, and similar such processes.

The present invention relates to using modified exosomes loaded with cargo for CVD. The present invention further relates to using modified exosomes associated with cargo for treating cancer. The present invention further relates to using modified exosomes associated with cargo for treating Atherosclerotic Cardiovascular Disease (ASCVD) and Chronic Heart Failure (CHF).

FIG. 1 illustrates a method for producing autologous exosomes from a body fluid according to an embodiment of the invention. Although the method 100 is illustrated and described as a sequence of steps, its contemplated that various embodiments of the method 100 may be performed in any order or combination and need not include all of the illustrated steps. The method 100 comprises the step of: collecting body fluid 110 from a subject, extracting exosomes 120 from the body fluid, modifying said exosomes 130, administering modified exosomes 140, and evaluating a health-related outcome 150.

In step 110, body fluid is collected from a subject. The subject may be a human or an animal. The body fluid can be peripheral blood, cerebral spinal fluid, secretions, or any other body fluid in which exosomes can be extracted.

In step 120, exosomes are extracted from the body fluid. The extraction method depends on a number of factors including the type of body fluid extracted. Peripheral blood, for example, contains peripheral blood mononuclear cells and cellular component layers that can be separated by centrifugation at a medical facility. During the extraction process plasma, cells and cellular components are kept on dry ice at all times before isolation.

In one embodiment, the body fluid is transported to a laboratory to undergo isolation. In one embodiment, exosome isolation is achieved using a gradient method or a designated isolation kit (i.e., Total Exosome Isolation kit, ThermoFisher). The isolation kit protocol is highly efficient in yielding high amounts of exosomes from a body fluid, a cell culture media, or cell.

The method 100, provides several approaches to further optimize isolation of exosomes and increase exosome yield from a body fluid. In one embodiment, a gradient column separates components of the collected peripheral blood by cell densities. Such cellular densities correspond to exosomes and exosome-related materials. Another embodiment uses an exosome sorting method, where sorting markers or sorting beads are used to isolate exosomes from solution. A further embodiment uses flow cytometry sorting, which uses surface biomarkers present on exosome to identify and sort exosomes and exosome-related materials from cells and cell suspensions. In one embodiment, an exosome can be modified to include a targeting agent on a surface of the exosome.

Specifically, the exosomes can be modified (modified exosomes) to have specific targeting agents on their surface. In various examples, the modified exosome may have a targeting agent covering an entire surface or a partial surface of the extracellular vesicle. Thin layer chromatography can be used to optimally separate exosomes and exosome-related products according to specific exosome-associated surface proteins and lipids. An exosome from peripheral blood, for example, would have exosome-related products such as transferrin receptors (immature exosomes), signaling molecules, and similar cellular components. In another embodiment, ionic separation by drift time can be used to optimize isolating exosomes. For example, mass spectrometry may be used to isolate high yields of exosome and exosome-related products on the order of Ion mobility spectrometry-mass spectrometry may also be performed when physicochemical properties of both the exosome and the cargo need to be defined prior to loading into the exosome.

Isolated exosome samples can be purified using column methods in accordance with cGMP protocols and regulatory requirements.

In step 130, the exosomes are modified by incorporating cargos. In one embodiment, the modifications to the exosomes are done ex vivo. The exosomes can be further modified to have on its surface. Exosomes are assembled or transfected with cargo using a number of methods. In one embodiment depending on the physicochemical properties of the load, the exosomes are assembled or transfected with cargo using liposomes (Lipofectamine 2000, Exofect, or heat shock). In another embodiment, exosomes are assembled or transfected with cargo using CAR-T cells transduced without using retroviruses. In another embodiment, exosomes are assembled or transfected with cargo using retroviruses, AdV, AAV of any variety and strain. In another embodiment, exosomes are assembled or transfected with cargo using DNA viruses, siRNA, long interference RNA, noncoding RNA, iRNA, RNA vectors. In another embodiment, exosomes are assembled or transfected with cargo using DNA, DNA plasmids, CRISPR, CRISPR/Cas9 and/or any endonucleases for gene editing. In another embodiment, exosomes are assembled or transfected with cargo using gene editing technology, small molecules, antibodies, and proteins including recombinant endogenous proteins. In another embodiment, exosomes are assembled or transfected with cargo using oligonucleotide therapeutics, including ASO, gene targeting technology, and gene correction technology. In another embodiment, exosomes are assembled or transfected with cargo using synthetic/molecular conjugates and physical methods for delivery of gene and cell therapeutics.

In step 130, the method for loading exosomes efficiently and effectively incorporates autologous or exogenous materials (therapeutic compounds above or any endogenous enzyme, protein, lipid, molecule, DNA or RNA of interest). In non-limiting examples, the method for loading an exosome can include the process of: 1) Lipid-lipid affinity, using liposomes of high and low density; 2) Incorporating intracellular affinity proteins and/or molecules into the exosome; 3) Using Clathrin coated vesicles in clathrin-mediated endocytosis methods for incorporation of a therapeutic molecule into an exosome or an exosome-like carrier; and 4) Endocytosis receptors/proteins methodology. In one embodiment the method for loading exosomes includes the methods of exosome membrane dissociation and reconstitution via chemical or electromagnetic gradient changes. In one embodiment, a method is used for large molecules or heavy compounds. In certain examples, the optimization of the method 100 is due to including transmembrane transporters activators when loading the biological materials into the exosomes. After the exosome has been loaded, any potential activator remaining in the exosome will be filtered and purified using column methods in compliance with cGMP and regulatory requirements before undergoing the next processing steps.

Exosomes loaded with cargo are considered mature exosomes and are inspected for cGMP compliance, purity and stability for quality assurance and quality check. Next, mature exosomes that have passed the quality check undergo an expansion process if needed. Next, the mature exosomes are diluted and premix into saline/vehicle (depending on the characteristics of the load) for a ready to administer tube/cartridge. Finally, the suspension is frozen and stored or shipped to a site for use in clinical or preclinical studies and to patients for self-injection of approved-clinical grade mature exosomes.

In step 140, the mature exosomes are administered to a subject. The subject, may be the same subject from which the body fluid was collected in step 110. The method of administering the exosomes 140 includes, but is not limited to: intravenous, intra-arterial, intra-thecal, intra-ventricular, subcutaneous, subdermal, oral, rectal, intra-peritoneal, transdermal, intraosseous injection, intraosseous infusion, or a combination thereof. In one embodiment the mature exosomes are administered in vivo.

In step 150, the outcome of the treatment is evaluated. This evaluation can be done using a variety of methods, which is immediately apparent to one of ordinary skill in the art.

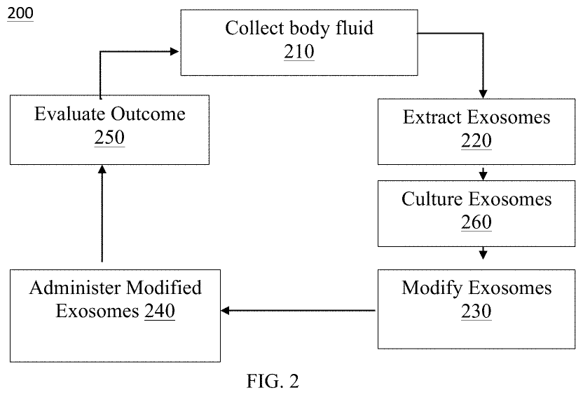

FIG. 2 illustrates a method for producing autologous exosomes from a body fluid according to an embodiment of the invention. Although the method 200 is illustrated and described as a sequence of steps, its contemplated that various embodiments of the method 200 may be performed in any order or combination and need not include all of the illustrated steps. The method 200 comprises the step of: collecting body fluid 210 from a subject, extracting exosomes 220 from the body fluid, culture the exosomes 260, modifying the exosomes 230, administering modified exosome 240, and evaluating the outcome 250.

In step 210, body fluid is collected from a subject. The subject may be a human or an animal. The body fluid can be peripheral blood, cerebral spinal fluid, secretions, or any other body fluid in which exosomes can be extracted.

In step 220, exosomes are extracted from the body fluid using methods as explained above.

In step 260 the exosomes are subjected to a primary culture and expansion. The exosomes will be extracted from primary cultured cells using a gradient or filtration method or a designated expansion kit (i.e., Total Exosome Isolation kit (from cell culture media), ThermoFisher). The cell culture and expansion may be frozen and stored for future exosome isolation procedures/protocols per the methods described above.

In step 230, the exosomes are modified by incorporating cargos. Exosomes are assembled or transfected with cargo using a number of methods as explained above. In one embodiment, the step of modifying the exosomes occurs ex vivo.

In step 240 the mature exosomes are administered to a subject using methods as explained above. The step of administering the modified exosomes can occur in vivo or in vitro.

In step 250, the outcome of the treatment is evaluated. This evaluation can be done using a variety of methods, which is immediately apparent to one of ordinary skill in the art.

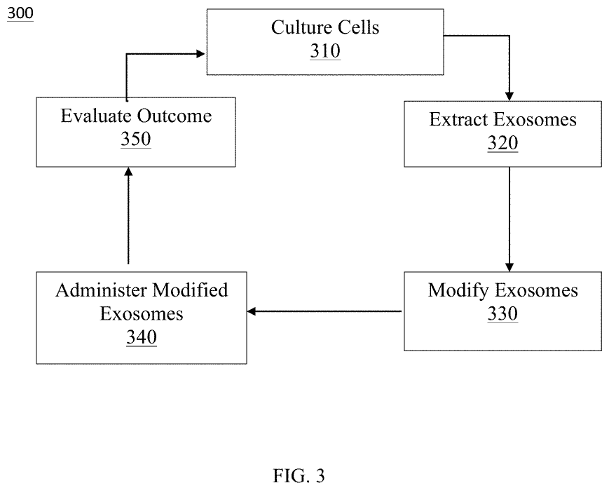

FIG. 3 illustrates a method for producing autologous exosomes from a cell culture according to an embodiment of the invention. Although the method 300 is illustrated and described as a sequence of steps, its contemplated that various embodiments of the method 300 may be performed in any order or combination and need not include all of the illustrated steps. The method 300 comprises the step of: culturing cells 310, extracting exosomes 320 from the cell culture, modifying the exosomes 330, administering modified exosome 340, and evaluating the outcome 350.

In step 310, primary or stable cell lines of human or animal origin are cultured and expanded with standard conditions.

In step 320, exosomes are extracted from the cultured cells.

In step 330, the exosomes are modified by incorporating cargos. Exosomes are assembled or transfected with cargo using a number of methods as explained above.

In step 340 the mature exosomes are administered to a subject using methods as explained above.

In step 350, the outcome of the treatment is evaluated. This evaluation can be done using a variety of methods, which is immediately apparent to one of ordinary skill in the art.

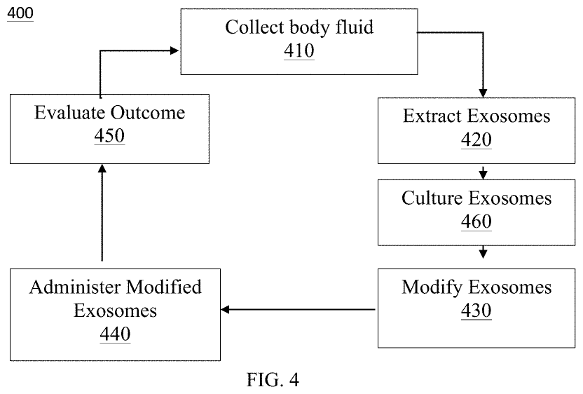

FIG. 4 illustrates a method for producing autologous exosomes from body fluid according to an embodiment of the invention. Although the method 400 is illustrated and described as a sequence of steps, it's contemplated that various embodiments of the method 400 may be performed in any order or combination and need not include all of the illustrated steps. The method 400 comprises the step of: collecting body fluid 410, extracting exosomes 420 from the body fluid, culturing the exosomes 460, modifying the exosomes 430, administering modified exosome 440, and evaluating the outcome 450.

In step 410, a body fluid is collected from a universal donor or patient. The subject may be a human or an animal. The body fluid can be peripheral blood, cerebral spinal fluid, secretions, or any other body fluid in which exosomes can be extracted.

In step 420, exosomes are extracted from the body fluid using methods as explained above.

In step 460, the exosomes are cultured. The exosomes are expanded using a primary cell culture from the body fluid of the universal donor or patient using a gradient method or a designated isolation kit (i.e., Total Exosome Isolation kit, ThermoFisher). The isolation kit protocol is highly efficient in yielding high amounts of exosomes from either body fluids or cell culture media or cell. The cell culture and expansion from the universal donor or patient may be frozen and stored for future exosome isolation procedures/protocols per the methods described above.

In step 430, the exosomes are modified by incorporating cargos. Exosomes are assembled or transfected with cargo using a number of methods as explained above.

In step 440 the mature exosomes are administered to a subject using methods as explained above.

In step 450, the outcome of the treatment is evaluated. This evaluation can be done using a variety of methods, which is immediately apparent to one of ordinary skill in the art.

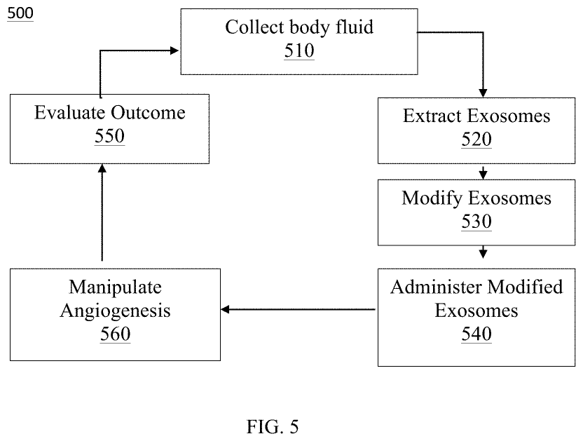

FIG. 5 illustrates a method for producing exosomes loaded with cargo for treating CVD or cancer according to an embodiment of the invention. Although the method 500 is illustrated and described as a sequence of steps, its contemplated that various embodiments of the method 500 may be performed in any order or combination and need not include all of the illustrated steps. The method 500 comprises the step of: collecting body fluid 510, extracting exosomes 520 from the body fluid, modifying the exosomes 530, administering modified exosome 540, manipulating angiogenesis 560, and evaluating the outcome 550.

In step 510, a body fluid is collected from a universal donor or patient. The subject may be a human or an animal. The body fluid can be peripheral blood, cerebral spinal fluid, secretions, or any other body fluid in which exosomes can be extracted.

In step 520, exosomes are extracted from the body fluid using methods as explained above.

In step 530, the exosomes are modified by incorporating cargo. The type of cargo is dependent on the desired effect on angiogenesis. In one embodiment, to help patients with illnesses such as CVD, promoting angiogenesis is desired. In such an embodiment, exosomes are loaded with antibodies for secreting VEGF to promote angiogenesis. The exosomes are assembled or transfected with cargo using a number of methods as explained above.

In another embodiment, to help patients with illnesses such as cancer, reducing angiogenesis is desired. In such an embodiment, exosomes are loaded with anti-miR214 to reduce angiogenesis. The exosomes are assembled or transfected with cargo using a number of methods as explained above.

In step 540 the mature exosomes are administered to a subject using methods as explained above.

In step 560, the angiogenesis is manipulated. In an embodiment, where the exosomes are loaded with antibodies for secreting VEGF, angiogenesis is promoted. In an embodiment, where the exosomes are loaded with anti-miR214, angiogenesis is reduced.

In step 550, the outcome of the treatment is evaluated. In an embodiment, where the exosomes are loaded with antibodies for secreting VEGF, the patient may be assessed for generated collaterals. A new medical regimen may be implemented depending on the results.

In an embodiment, where the exosomes are loaded with anti-miR214, the patient may be assessed for tumor growth, metastasis, and/or malignancy. A new medical regimen may be implemented depending on the results.

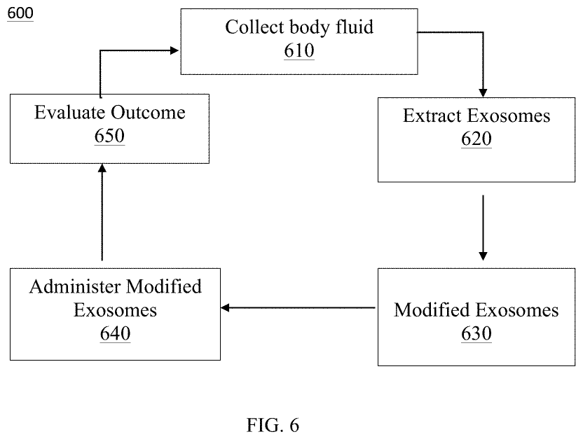

FIG. 6 illustrates a method for producing exosomes loaded with cargo for treating CVD according to an embodiment of the invention. Although the method 600 is illustrated and described as a sequence of steps, its contemplated that various embodiments of the method 600 may be performed in any order or combination and need not include all of the illustrated steps. The method 600 comprises the steps of: collecting body fluid 610, extracting exosomes 620 from the body fluid, modifying the exosomes 630, administering modified exosome 640, and evaluating the outcome 650.

In step 610, a body fluid is collected from a universal donor or patient. The subject may be a human or an animal. The body fluid can be peripheral blood, cerebral spinal fluid, secretions, or any other body fluid in which exosomes can be extracted.

In step 620, exosomes are extracted from the body fluid using methods as explained above.

In step 630, the exosomes are modified and loaded with antibodies secreting PCSK-9, IL1b, IL4, or IL13 to reduce angiogenesis. The exosomes are assembled or transfected with cargo using a number of methods as explained above.

In step 640 the mature exosomes are administered to a subject using methods as explained above.

In step 650, the outcome of the treatment is evaluated. This evaluation can be done using a variety of methods, which is immediately apparent to one of ordinary skill in the art.

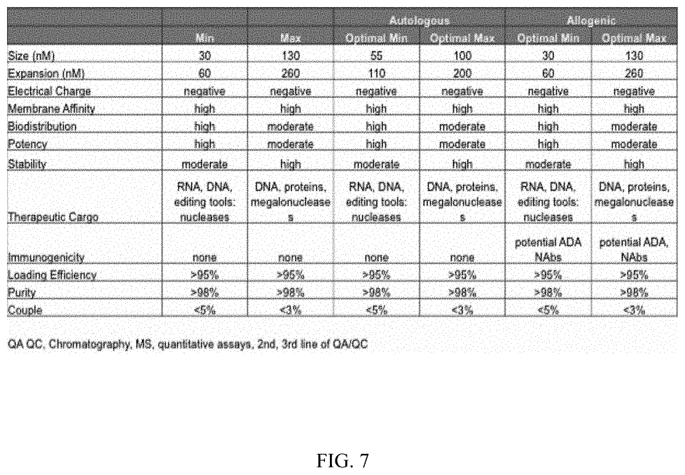

FIG. 7 illustrates the parameters used to sort exosomes according to an embodiment of the invention. In one embodiment, the invention provides autologous exosomes having an optimal vesicle size between about 55 nanometers (nM) and 100 nM. In certain embodiments, allogenic exosomes have an optimal vesicle size between about 30 nM and 130 nM. A vesicle size between 55 nM and 100 nM may be chosen as larger exosomes are less stable. Also, larger exosomes can couple with other exosomes making calculating drug dose, bioavailability, and biodistribution challenging. In some embodiments, the exosomes have the ability to expand to a size between about 60 nM and 260 nM. Such expanded exosomes can encompass large constructs. In some embodiments, the expanded exosomes can encompass more than or equal to about 7 kilo bases (Kb), and accommodate one or more copies of a relatively large viral particle such as an AAV. In one example, an exosome is loaded with at least four AAV particles to improve an exosome safety profile. In some embodiments, either an autologous or allogenic exosome has a negative electrical charge. Both autologous and allogenic exosomes can have a high membrane affinity. In some embodiments, biodistribution is moderate to high. Potency can range from high to moderate. Stability is moderate to high.

In an embodiment, exosomes can comprise a smaller sized cargo comprising RNA, DNA, editing tools (e.g., nucleases), or any combination thereof. In another embodiment, an exosome can comprise a larger cargo comprising DNA, proteins, megalonucleases, or a combination thereof. One advantage of autologous exosomes is that they do not illicit a significant immune response. Allogenic exosomes may illicit anti-drug antibodies (ADA) and neutralizing anti-bodies (NAb). One embodiment of the present invention enables high efficiency of loading cargo into at least ninety-five percent (95%) of exosomes. Another embodiment can provide a higher purity of exosomes of at least ninety-eight percent (98%).

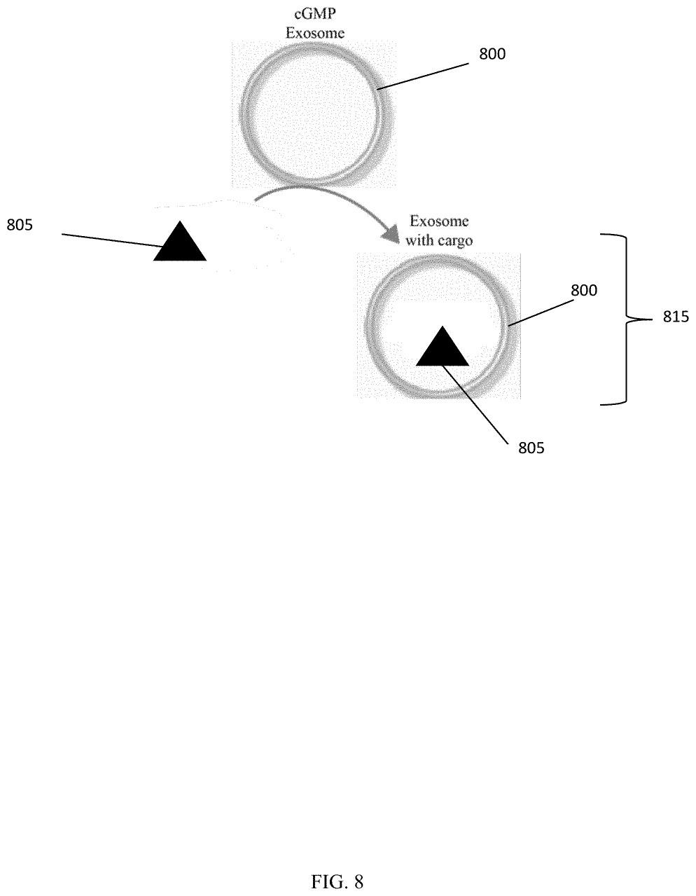

FIG. 8 illustrates an exosome 800 loaded with cargo according to an embodiment of an invention. The different methods for isolating the cGMP exosome 800 have been described above. In one embodiment, the cGMP exosome 800 is loaded with cargo 805. The resulting mature exosome 815 is inspected for cGMP compliance, purity and stability for quality assurance and quality check. As discussed above, the mature exosomes, that have passed the quality check, may undergo an expansion process. In an embodiment, the mature exosomes are diluted and premix into saline or a similar such solution for a ready to administer tube. Finally, the suspension can be frozen and shipped to a site for use in clinical or preclinical studies and to patients for self-injection of approved-clinical grade mature exosomes.

In one embodiment the cargo 805 is plasmid secreting antibodies, wherein the plasmid is an RNA plasmid, an RNAi plasmid, a DNA plasmid, an iDNA plasmid, or a combination thereof. In one embodiment, the cargo is a PCSK9 iRNA plasmid secreting antibodies.

In another embodiment, the exosome 800 is loaded with cargo 805, which is VEGF protein, antibodies for secreting VEGF, or a derivative thereof. In certain cases, the cargo 805 comprises at least [N] copies of VEGF protein, antibodies for secreting VEGF, or a derivative thereof.

In another embodiment, the exosome 800 is loaded with cargo 805, which is anti-miR214 or a derivative thereof. In certain cases, the cargo 805 comprises at least [N] copies of anti-miR214 or a derivative thereof.

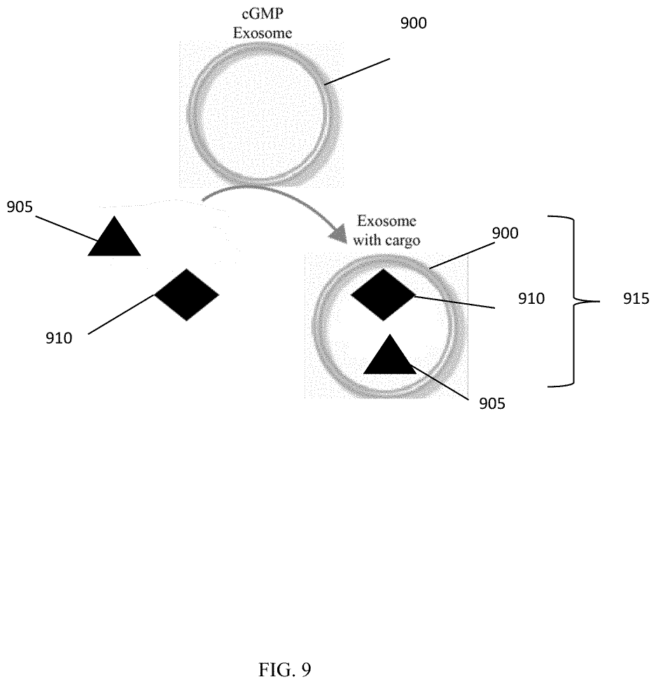

FIG. 9 illustrates a schematic of an exosome 900 comprising a first cargo 905 and a second cargo 910 according to an embodiment of an invention. The first cargo 905 and second cargo 910 may be plasmid secreting antibodies, wherein the plasmid is an RNA plasmid, an iRNA plasmid, a DNA plasmid, an iDNA plasmid, or a combination thereof. In another embodiment the first cargo 905 and second cargo 910 may be proteins used to treat an illness. The different methods for isolating the cGMP exosome 900 have been described above. In one embodiment, the cGMP exosome 900 is loaded with the first cargo 905 and the second cargo 910. The resulting mature exosome 915 is inspected for cGMP compliance, purity and stability for quality assurance and quality check. As discussed above, the mature exosomes, that have passed the quality check, may undergo an expansion process. In an embodiment, the mature exosomes are diluted and premix into saline or a similar such solution for a ready to administer tube. Finally, the suspension can be frozen and shipped to a site for use in clinical or preclinical studies and to patients for self-injection of approved-clinical grade mature exosomes.

In this embodiment two cargos are shown but, any number of cargos discussed may be loaded into a single exosome. Further, the different types of cargo may be loaded into the exosome 900 in any number of combinations. In one embodiment, the exosome 900 may have two or more cargos wherein the two or more cargos may be identical or substantially the same. In another embodiment, an exosome may have two or more cargos wherein each of the two or more cargos are distinct from one another.

In one embodiment the first cargo 905 is plasmid secreting antibodies, wherein the plasmid is an RNA plasmid, an iRNA plasmid, a DNA plasmid, an iDNA plasmid, or a combination thereof. In said embodiment, the second cargo 910 is a PCSK9 iRNA plasmid secreting antibodies.

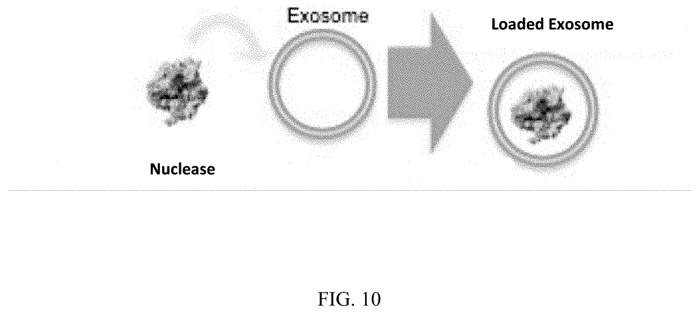

FIG. 10 illustrates a cGMP grade-exosome loaded with cargo comprising a nuclease according to an embodiment of the invention. The nuclease functions as a base editor to correct or manipulate the targeted single nucleotide polymorphisms (SNPs). In one embodiment, in vitro, an exosome mediated-nuclease delivery enables a nuclease to correct the base mutation of cells carrying certain SNPs. In specific examples, a loaded exosome comprising a nuclease base editor is delivered to cells that may be effected by a particular defect. Base editors are the latest generation of gene editing tools with very high precision at targeting single nucleotides within a sequence. In one embodiment, the loaded exosomes meeting clinical-grade GMP, regulatory chemistry manufacturing and controls (CMC) compliance can be deployed to patients that suffer from CVD, coronary arterial disease, atherosclerotic CVD, peripheral arterial disease, peripheral vascular, cancer, and similar such illnesses.

Base editors show very low (0.1%) indel formation (insertion or deletion of bases in the genome), which makes it safe for therapeutic use. A nuclease base editor enables treating certain illnesses by targeting and correcting one or both alleles at a particular DNA sequence. The use of such a nuclease base editor is apparent to one of ordinary skill in the art. A nuclease base editor corrects one or both alleles at a particular DNA sequence.

The proportion of loading is 1:1 (exosome: base editor) using proprietary techniques that include electromagnetism and membrane dissociation technologies. In one embodiment, exosomes having a vesicle size between fifty-five (55) and one hundred (100) nM are selected for cargo loading.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. A composition for delivering cargo to cytoplasm of a cell, wherein the cargo manipulates angiogenesis, the composition comprising:
an exosome; and
cargo, located within the exosome, comprising at least one plasmid.

2. The composition of claim 1, wherein the exosome is isolated from autologous cells of a patient.

3. The composition of claim 1, wherein the exosome is isolated from a cell line, a primary cell culture, or a combination thereof.

4. The composition of claim 1, wherein the exosome is isolated from a stem cell.

5. The composition of claim 1, wherein the at least one plasmid is a RNA plasmid, a DNA plasmid, or any combination thereof.

6. The composition of claim 1, wherein the at least one plasmid is a retrovirus or an adeno-associated virus (AAV).

7. The composition of claim 1, wherein the at least one plasmid promotes expression of the VEGF gene.

8. The composition of claim 1, wherein the at least one plasmid is a proprotein convertase subtilisin/kexin type 9 inhibitor.

9. The composition of claim 1, wherein the at least one plasmid is a miR-214 inhibitor.

10. The composition of claim 1, wherein the cargo further comprises a CRISPR-Cas9 system, a Zinc finger, a single base editor, or a combination thereof.

11. The composition of claim 1, wherein the at least one plasmid is a DNA plasmid bioengineered specifically to self-produce monoclonal neutralizing antibodies.

12. The composition of claim 1, wherein the exosome further comprises at least one targeting agent.

13. The composition of claim 12, wherein the at least one targeting agent is a protein epitope.

14. A composition for delivering cargo to cytoplasm of a cell, wherein the cargo manipulates angiogenesis, the composition comprising:
an exosome; and
cargo, located within the exosome, comprising a DNA plasmid bioengineered specifically to self-produce monoclonal neutralizing antibodies.

15. The composition of claim 14, wherein the cargo further comprises a CRISPR-Cas9 system, a Zinc finger, a single base editor, or a combination thereof.

16. The composition of claim 14, wherein the cargo further comprises siRNA.

17. The composition of claim 14, wherein the DNA plasmid further comprises a promoter.

18. The composition of claim 14, wherein the cargo further comprises at least one plasmid, which promotes the expression of the VEGF gene.

19. The composition of claim 14, wherein the exosome further comprises at least one targeting agent.

20. The composition of claim 19, wherein the at least one targeting agent is a protein epitope.
